(19) United States
(12) Patent Application Publication
(10) Pub. No.: US 2024/0309325 A1
(43) Pub. Date: Sep. 19, 2024

(54) Nature and formation mechanism of red blood cells and platelets and the mechanism of new artificial blood

(71) Applicants: Wendy Wei Sun, Hockessin, DE (US); Derui Huang, Hockessin, DE (US); Yanhe James Huang, Hockessin, DE (US)

(72) Inventors: Wendy Wei Sun, Hockessin, DE (US); Derui Huang, Hockessin, DE (US); Yanhe James Huang, Hockessin, DE (US)

(21) Appl. No.: 18/123,316

(22) Filed: Mar. 19, 2023

(51) Int. Cl. C12N 5/078; C12N 5/00

(52) U.S. Cl. CPC C12N 5/0641; C12N 5/0006; C12N 5/0644; C12N 2500/35

(57) ABSTRACT

This patent proves that enucleated red blood cells are the product of biological evolution and disabled red blood cells due to functional defects. Platelets are the nuclei removed during the enucleation of nucleated red blood cells. They are useful components for blood coagulation and hemostasis, but not essential components. Nucleated erythrocytes are fully functional erythrocytes. The preparation of nucleated red blood cell blood, especially the universal blood without blood type, not only has all the functions of red blood cells, but also can be replicated in the blood, improving the physiological performance of blood and reducing the cost and time of artificial blood.

### TECHNICAL FIELD

The invention belongs to the discovery of the formation mechanism of blood red blood cells and platelets and the invention of artificial blood.

### BACKGROUND OF THE INVENTION

At present, the demand for medical blood is increasing day by day, and the blood supply is still mainly based on volunteer donations. Artificial blood is still in the laboratory stage due to the long process and high cost. Research on the formation mechanism of red blood cells and platelets and simplifying the preparation of artificial blood has become the most urgent topic today.

### SUMMARY OF THE INVENTIONS

Nucleated red blood cells are not the cause of anemia in mammals, but insufficient hemoglobin is the main cause of anemia. Enucleated erythrocytes are not the best structure for blood oxygen transmission. Enucleated erythrocytes are passive evolution products of erythrocyte replication failure during the evolution from non-mammals to mammals but still retain the main physiological functions. Enucleated erythrocytes are disabled erythrocytes and should not be mature red blood cells. Platelets are the nuclei that are removed during the enucleation of nucleated red blood cells which play a physiological role as a hemostatic coagulation factor. Platelet is a useful factor for hemostasis and coagulation, but it is not an essential factor, and its hemostasis and coagulation function can be replaced by other cells in the blood. The nucleus of red blood cells is non-toxic, and nucleated red blood cells can not only play the role of enucleated red blood cells, but also produce more nucleated red blood cells in the blood through reproducing and proliferating with mitosis, and relieve the pressure of bone marrow hematopoietic tissue, especially in patients with hematopoietic diseases. The production of artificial blood with nucleated red blood cells but without components of platelets can avoid bacterial and virus contamination, greatly reduce the production cycle and cost of artificial blood, meet the needs of medical blood supply shortages, and reduce the demand for blood donation. Removing blood type control genes and preparing nucleated red blood cells without blood type antigens can avoid the risk of blood type configuration and hemolysis during blood transfusion and remit the need for patients with rare blood types.

### DETAILED DESCRIPTION OF THE INVENTION

### 1. Introduction to Blood

Animals are divided into invertebrates and vertebrates; Invertebrate blood has few blood cells and no red blood cells. Certain invertebrates possess heme, such as worm red blood protein, which can carry oxygen. Some invertebrates have respiratory pigments for binding oxygen, which are either dissolved in hemolymph or packaged in hemolymphocytes to carry oxygen.

When invertebrates evolved into vertebrates, the blood in vertebrates had more and more powerful blood cells, including nucleated red blood cells (RBC) and white blood cells.

When some vertebrates evolved into mammals, the nucleated red blood cells in the body evolved into enucleated red blood cells (RBC), added platelets, and blood cells became three types of cells: enucleated red blood cells, white blood cells, and platelets. So, are nucleated and enucleated red blood cells related to platelets?

Blood is a red, opaque and viscous liquid. It flows in the blood vessels and heart of organisms, and performs five basic functions of transportation, regulation of body temperature, defense, regulation of body osmotic pressure and acid-base balance. It brings oxygen and nutrients to living cells and removes metabolites produced by cells including carbon dioxide. Blood is composed of plasma and blood cells, and the function of blood includes two parts: plasma function and blood cell function.

Plasma contains serum, coagulation factors and water. Serum contains proteins such as albumin and globulin. Coagulation factors include fibrin and blood cells. Among blood cells, red blood cells (including nucleated and enucleated red blood cells) are involved in transporting the oxygen needed by the human body and excreting metabolite carbon dioxide; white blood cells are immune cells that fight infection and are mainly involved in the immune function of the body; platelets are coagulation factors that can stop damaged blood vessels bleeding and cause tissue clotting. Modern medicine also refers to enucleated red blood cells as normal mature red blood cells, and believes that enucleated red blood cells can transport oxygen more efficiently.

This patent mainly relates to red blood cells and platelets.

### 2. Red Blood Cell Characteristics, Modern Mechanism of Red Blood Cell Formation and Enucleation

When the body is hypoxic, it will promote the production of erythropoietin by the kidneys. This hormone can stimulate the proliferation and differentiation of red blood cell precursor cells, and eventually develop into mature red blood cells in hematopoietic tissues. In postnatal birds and mammals (including humans), the red bone marrow is the organ that produces red blood cells. Before the age of 5, all bone marrow produces red blood cells throughout the person's body. By about 25 years of age, the level of hematopoiesis in the tibia and femur will drop to a very low level. The spine, sternum, pelvis, ribs, and skull continue to produce red blood cells throughout a person's life.

Red blood cells (RBCs, or erythrocytes) are the most numerous blood cells in the blood, with an average diameter of 9 μm.

The main functional molecules of red blood cells are hemoglobin and iron. 90% of red blood cell protein is composed of hemoglobin, water accounts for 64%, and the remaining 4% is fat, sugar and various electrolytes. Hemoglobin is a protein molecule containing heme, which is very active and can bind both oxygen and carbon dioxide. Therefore, its main job is to transport oxygen and carbon dioxide. It is the most important medium in vertebrates to transport oxygen to various tissues of the body through the combination of blood hemoglobin and oxygen molecules in the lungs or gills, and then release the combined oxygen molecules in the tissues of the body. Oxygen molecules can easily diffuse through the cell membrane of red blood cells. Hemoglobin also transports carbon dioxide produced by the organism using oxygen (less than 2% of total oxygen, more carbon dioxide is transported by plasma). The function of red blood cells is also to transport electrolytes, glucose and amino acids, which are necessary for human metabolism.

Biologists believe that birds are non-mammalian vertebrates and have no bone marrow. In order to be able to fly, their bones are hollow. There are no bone marrow hematopoietic stem cells, and they cannot form blood cells in the blood. Bird red blood cells are nucleated red blood cells, their red blood cells can go through mitosis to maintain the number to meet activity requirements, which is a need for the survival of birds.

Mammalian red blood cells do not have nuclei. They are formed by multiple differentiations of bone marrow stem cells, including the process of depleting the nucleus. The specific reason of enucleation is not clear, and saving space is one of the explanations. After our red blood cells are enucleated, it means that they have lost their DNA, and there are no organelles such as Golgi apparatus and mitochondria, so they cannot produce structural proteins, repairing proteins or enzymes, which make them with a limited lifespan and will age in 70-140 days. Because its aging and death will easily lead to blood vessel blockage, it will automatically return to the depths of the bone marrow for destruction by white blood cells; or when it passes through the liver, it will be engulfed and decomposed by macrophages to become bile. The components of blood will also be completely replaced within a certain cycle. However, enucleated red blood cells still have metabolic functions. They break down glucose through the glycolysis pathway to release energy, which helps enucleated red blood cells to run. Moreover, there are no insulin receptors on the surface of red blood cells, so their sugar intake cannot be regulated by insulin. This means that glucose can easily enter the red blood cells.

Mammalian erythrocytes are flat, with the center of both sides sunken inwards. This shape maximizes oxygen uptake from the surroundings. It is also flexible, which allows it to pass through capillaries and release oxygen. Red blood cells in mammals are basically round, except in camels where they are oval.

Blood cells are derived from pluripotent mother cells. Hematopoietic stem cells (HSC) are a group of initial cells that are extremely small in number, capable of self-renewal and capable of generating various blood cells and immune cells, most of which exist in a resting state in the bone marrow. When the body is stimulated by the outside world, HSC can be rapidly activated and then proliferate and differentiate into downstream mature cells to maintain the hematopoietic homeostasis of the body. The uncontrolled regulation or disease of HSC will cause a variety of blood diseases. Hematopoietic stem cells give rise to lymphoid stem cells and bone marrow stem cells. Bone marrow stem cells are the progenitor cells that give rise to erythrocytes, granulocytes, monocytes and megakaryocytes. The process by which progenitor cells differentiate into red blood cells is called erythropoiesis.

According to modern medicine, the production of normal human erythrocytes includes: the stage of hematopoietic stem cells, the stage of erythroid progenitor cells, the proliferation and differentiation stages of erythroid precursor cells (primary erythrocytes to late erythrocytes), the proliferation and maturation process of reticulocytes, and the release of mature red blood cells from reticulocytes into the peripheral blood. The process by which erythrocytes release mature red blood cells into the peripheral blood. When red blood cells are produced in the bone marrow, they have nuclei. Medically, nucleated red blood cells are called immature red blood cells. A small amount of nucleated red blood cells can be seen in the peripheral blood of newborns within one week of birth which cannot be seen in the peripheral blood of normal adults. If nucleated red blood cells appear, it is a pathological phenomenon. The anemia status of the patient can be judged by detecting the proportion of nucleated red blood cells.

The process by which nucleated red blood cells lose their nucleus during their maturation is called enucleation. The process of cell maturation is a process of increasing hemoglobin and attenuating nuclear activity. During the process of nuclear activity attenuation, hemoglobin is also increasing continuously. When the nuclear function completely disappears, the amount of hemoglobin reaches the highest amount. The increase of hemoglobin in red blood cells promotes the loss of nuclear activity which no longer synthesizes DNA or RNA. This is because hemoglobin enters the nucleus through the internuclear pores and acts on nuclear histones, leading to chromosome inactivation and promoting nuclear condensation. The late immature erythroblasts have lost the ability to continue to divide, the nucleus is condensed and solidified, and it is biased towards the one pole of the cell membrane and loses transcriptional activity. The late red blood cell increases its own wave-like movement and then undergoes several contractions to squeeze the nucleus to the cytoplasmic pole. Then actin wraps around the cell membrane in a ring and shrinks, thereby dividing the nucleus from the cell, and the nucleus is also wrapped in cytoplasm. The excised nuclei are subsequently phagocytized by macrophages, or fragmented and lysed within the spleen, or enter the peripheral blood system directly. The remainder of the cytoplasm forms mature red blood cells, which are released into the blood system. The significance of the denuclearization of red blood cells is that after removing the large proportion of the nucleus, it can form a “biconcave disc-like” shape that is most conducive to carrying oxygen, and it can easily change the cell's shape to pass through fine structures such as capillaries. This is the modern mechanism of red blood cell enucleation.

Modern medicine believes that nucleated red blood cells are immature red blood cells. The number in the peripheral blood can reflect the production function of bone marrow red blood cells and is an important indicator for judging the curative effect of anemia and related diseases. Therefore, it is important for the diagnosis of blood diseases and the observation of treatment responses.

Red blood cell survival (RBCS) refers to the survival time of red blood cells released from bone marrow to peripheral blood. The normal value of RBCS is 120 days, and the range is 70 to 140 days. Birds are lower vertebrates, and all other vertebrates except mammals are nucleated red blood cells with a life span of 25-30 days. This patent believes that the short lifespan of nucleated red blood cells is not because the nucleated red blood cells are prone to aging, but that they multiply by mitotic differentiation, and the number of nucleated red blood cells is multiplied by division.

Through erythropoiesis, 2-3 million new red blood cells are produced in the human body every second and released into the circulatory system through the bone marrow. Anemia is caused by decreased levels of hemoglobin in RBCs in the circulatory system. Genetic defects in hemoglobin can also lead to diseases such as sickle cell anemia.

The relationship between red blood cell number, size, and hemoglobin content was investigated in 441 species of mammals, birds, and reptiles (101 families). Mammals have more and smaller red blood cells per unit volume of blood than birds, and birds have more and smaller red blood cells than reptiles. The reason may be that smaller erythrocytes increase the blood oxygen transport capacity and the exchange efficiency of hemoglobin and oxygen, because the smaller the cells, the larger the total surface area with the same mass of blood cells.

In bird blood cells, the red blood cells are nucleated, and there is a certain amount of water in the cells, which makes their homeostasis better. Birds that are good at flying have both more and smaller red blood cells than those that are not. Bird hemoglobin has a higher oxygen-carrying capacity. The biconcave shape of enucleated cells does not allow more surface area for oxygen diffusion than spherical cells, and non-mammalian red blood cells such as birds have nuclei, and does their nucleus not affect the passage of cells through capillaries? But deflated red blood cells indeed easily exchange oxygen and carbon dioxide due to lack of water. This shows that the presence or absence of a nucleus has no direct and huge impact on the oxygen-carrying capacity of red blood cells. It also shows that the red blood cell nucleus is not the factor that hinders the red blood cell to carry oxygen, and there should be other reasons for the enucleation.

### 3. New Mechanism of red Blood cell Formation and Enucleation

Why do mammalian red blood cells have no nucleus, but non-mammalian red blood cells such as birds do? Since non-mammals such as birds existed long before mammals, it's not why they have nuclei, but why non-mammals don't enucleate like mammals. What caused non-mammals to evolve into mammals? At present, the medical community is not very clear about the mechanism of red blood cell enucleation.

This patent thinks that red blood cell nuclei are obviously non-toxic, since they are non-toxic, why can't they have nuclei? Is it just because of weak oxygen carrying capacity?

If the red blood cells of mammals including humans have no nucleus and form a concave disc to increase the area to carry more oxygen, but birds consume more oxygen when flying, but the red blood cells have a nucleus and are oval, which is Why? And Birds that fly better have more red blood cells. Modern medical experts believe that birds naturally do everything possible to reduce the weight of their bodies after flying in the air for a long time, and the hollowness of the bones is an adaptation and a balance. We know that human red blood cells are developed from primitive red blood cells in the bone marrow through four cell mitosis. While birds have no bone marrow and hematopoietic stem cells, it increases the number of red blood cells only through the mitosis of red blood cells, so Birds sacrifice the possibility of increasing oxygen carrying. The bird red blood cells are oval, have a nucleus, and the volume is relatively large, generally 150 um³.

According to this patent, the reason for the hollow bones of birds is that bird red blood cells can produce enough red blood cells through mitotic replication, increasing the number of red blood cells to increase the oxygen-carrying capacity. However, human red blood cells do not have the function of mitotic replication to produce enough red blood cells, so they reversely stimulate hematopoietic tissues such as bone marrow, making the bone marrow and other hematopoietic tissues extra plump.

Human cell replication goes through mitosis. Mitosis and amitosis are manifested in different cells, only eukaryotic cells can undergo mitosis. It is the process by which a eukaryotic cell divides the chromosomes in its nucleus into two daughter nuclei after duplication. After the division of the nucleus, the nuclear chromosomes dominate the nutrient components of the cell to form new organelles, cytoplasm, and cell membrane components. These components diffuse to the periphery of the nucleus to form two independent new cells.

This patent believes that the essence of mitosis is the formation of a large amount of fibrin in the nucleus during division, which is usually called spindle filaments or microtubules. If a large amount of glycogen is formed at the same time, glycogen and fibrin form amyloid, and numerous amyloids that are organized in an orderly manner will reflect white frosty filaments visible under the microscope. This division is called mitosis. The disappearance of the spindle may be due to the reduction of fibrin and glycogen, the amyloid reversibly disappears, and the microscope cannot see it. If little or no glycogen is formed, adequate amounts of amyloid are not formed despite substantial fibrin formation, and no distinct white filamentous morphology is formed, it is said to be amitotic.

The process by which eukaryotic cells undergo mitosis varies between species. For example, animals cells, including human cells, undergo “open” mitosis, in which the nuclear envelope ruptures before chromosomes divide. Fungi carry out “closed” mitosis, in which chromosomes split into two daughter nuclei in the complete nuclear envelope.

The process of mitosis is highly complex and regular. During mitosis, chromatin forms chromosome pairs and is pulled by microtubules called spindle filaments, which drag the pairs of chromatids to the two poles of the cell. The cell then enters cytoplasmic division, producing two cells with the same genetic composition.

When cells enter mitosis from interphase, the microtubule network of interphase cells depolymerizes into free αβ-tubulin dimers, and then reorganizes into spindles to mediate the movement of chromosomes; at the end of division, the spindle microtubules depolymerize, which recombine to form the cytoplasmic microtubule network.

The new mechanism of cell replication in this patent: Cell replication originates from the replication of chromosomes into two groups, which are pulled by the spindle and split to the two poles. Chromosomes begin to play the role of genetic material, guiding cell nutrients to form new organelles, proteins, nuclear membranes, cell membranes, cytoplasm, etc. These new substances diffuse to the periphery of the newly formed nucleus, just like papermaking, depositing on the periphery, accumulating thicker, forming the nuclear membrane, cell membrane, cell wall, naturally forming a separation between the two nuclei, forming two new cell prototypes with the nucleus as the core are usually accompanied by cytokinesis, and the cytoplasm, organelles, and cell membranes are equally distributed to daughter cells, and the connection function between the two cell membranes gradually shrinks, blocking the relationship between the new cell and the outside world, the two new cells correspondingly divide independently.

The new mechanism of red blood cell enucleation in this patent: Human cell division begins after the nuclear membrane ruptures. Before the red blood cells enter the replication division, the hemoglobin concentration will gradually increase, and it will also be distributed in the nucleus area where the nuclear membrane ruptures. The blood sugar content of mammalian hematopoietic tissue is too high, and at the same time, the demand for glucose is too much, and it is easy to accumulate in the nucleus to form glycogen. The increase of hemoglobin concentration is conducive to the formation of amyloid between hemoglobin and glucose glycogen, and the amyloid leads to the gradual degeneration, reduction, and disappearance of some organelles (such as mitochondria, Golgi apparatus, and polyribosomes) used to synthesize hemoglobin, matrix proteins, and various enzymes in cells, and then block the conversion of nutrients into energy, resulting in the loss of energy in the nucleus live. Amyloid also binds and entangles DNA, RNA, and proteases that have not yet replicated and split, resulting in the failure of cell nucleus replication, which goes through dehydration, solidification and inactivation of the nucleus, and finally moves to the cell membrane and separates from the cytoplasm, resulting in cell enucleation, and the remaining cytoplasm enters the blood system as enucleated mature red blood cells.

This patent believes that there is a large amount of glucose around the hematopoietic bone marrow cells of mammals. If a virus or other factors cause metabolic rebalance, a larger amount of glucose will also be accumulated, and the glucose will form glycogen. Viruses or other factors can also promote the formation of short-chain polypeptides. Wdr26 induces and catalyzes the formation of polypeptides through nucleoprotein hydrolysis. Glycogen and these polypeptides form amyloid together. Amyloid wraps around nucleoproteins, nucleic acids and nuclei to condense and solidify them. The nuclear condensation, does not mean that nuclear proteins have leaked out of the nucleus, it is more likely that the nucleus is dehydrated and solidified. Hematopoietic bone marrow cells can usually divide and replicate, but in mammals, including humans, during the process of hematopoietic bone marrow cell replication and division, the nucleus has been condensed on one side of the cell or lysed out of hematopoietic bone marrow cells before it replicates and divides into two nuclei, which entered into the state of nuclear apoptosis, forming the enucleated red blood cells.

During nuclear condensation, neither new DNA nor new nuclear protein can be formed. The bound nucleic acid and nuclear protein cannot absorb water, and the nucleus dehydrates and shrinks. The binding of amyloid to nucleic acid makes the function of nuclear preparation protein atrophy, and the contents of the nucleus are correspondingly reduced to form nuclear atrophy, which in turn leads to enucleation.

The process of erythrocyte enucleation may also be referred to as erythrocyte nuclear atrophic apoptosis. In the enucleation, the chromatin of the erythrocyte nucleus is condensed and marginalized, and its chromatin DNA is broken. The atrophic erythrocyte nucleus is wrapped by the cell membrane to form apoptotic bodies, which bud off from the cell surface and are finally phagocytized by cells with phagocytic functions such as macrophages and epithelial cells.

Most scholars divide the form of cell death into three types: apoptotic programmed cell death (Apoptosis), autophagic programmed cell death (Autophagy) and cell necrosis (Necrosis).

This patent believes that the so-called programmed cell death is that the various functional parts of the cells cannot coordinate and work after aging to different degrees, resulting in cell aging death. This patent calls this kind of death as cell aging death, also known as aging apoptosis.

According to this patent, the aging of cells is the slow formation of amyloid in cells, which blocks the activity and function of nucleic acid and protease, leading to cell death. Erythrocyte enucleation is a special type of erythrocyte apoptosis characterized by nuclear pyknosis followed by enucleation. It is noninflammatory characterized with cellular pyknosis.

The key feature of this physiological (apoptotic) death process is the preservation of the integrity of the plasma membrane, as is the case with enucleated erythrocytes.

After the red blood cell is enucleated, it loses the chance of DNA replication and the possibility of protein synthesis. The area of the red blood cell membrane and the amount of hemoglobin will not increase, and the content of the red blood cell will no longer increase. In the blood flow, the red blood cells will naturally maintain a shape of concave in center and bulging at edge due to blood pressure factors. This is because the cell membrane of red blood cells has a certain thickness. When the nucleus is protruded, under the pressure of external blood pressure, the concave in center and bulging at edge is its natural state. Therefore, the inward depression and flat shape of the center of the enucleated red blood cells is not because it is conducive to carrying oxygen, but because of the physical state caused by blood pressure after enucleation.

This patent believes that the formation of so-called mature red blood cells is actually a disabled red blood cell, which makes the red blood cell lose its replication function and greatly reduces the lifespan and function of the red blood cell. This patent believes that immature erythrocytes can be replicated. We may not be able to change the derivation procedure and environment of human erythrocytes, but we can culture nucleated erythrocytes artificially. By controlling the environmental concentration of sugar, we can satisfy the speed of erythrocyte culture and keep erythrocytes at a nuclear stage, which will enhance the function of red blood cells and improve the situation of blood shortage.

According to this patent, anemia is due to the low diet or absorption capacity of the human body, the decrease in the concentration of blood sugar and related essential components, and the lack of nutrients that provide cell growth. That the slow growth of red blood cells and the stress response prompts the blood cell delivery system to quickly push immature (Nucleated) cells enter the blood system, leading to a decrease in the probability of red blood cell maturation, and an increase in the concentration of nucleated immature red blood cells entering the peripheral blood.

This patent believes that reticulocytes and nucleated red blood cells should not be the cause of anemia, but because anemia stimulates the hematopoietic system to rapidly replicate red blood cells, so that red blood cells are delivered into the peripheral blood system before entering the stage of replication and division. The manifestation is that the person is anemic, and there are too many reticulocytes and nucleated red blood cells in the blood. In fact, it is the inefficiency of blood transmission caused by the insufficient amount of effective hemoglobin. This patent thinks this mislead doctors and patients.

This patent believes that mammalian red blood cells are without nuclei, but cells cannot grow without nuclei, so nucleated erythrocytes with growth ability are actually regarded as immature erythrocytes by modern medical circles, in fact the so-called mature red blood cells are supposed to be disabled cells that have lost many functions.

According to this patent, nucleated cells are normal cells, and enucleation means that during the proliferation of nucleated cells, the nucleus is covered by super-strong spindle microtubules, inactivated and separated from the cytoplasm to detach from the cell. The remaining cytoplasm enters the blood system and becomes an enucleated red blood cell, which is a disabled red blood cell that has lost the ability to replicate.

This patent believes that the driving force of animal evolution lies in the abundance and persistence of blood sugar.

This patent believes that some animals have evolved from invertebrates to vertebrates, and their ability to grab food has made a qualitative leap. Red blood cells, oxygen-carrying cells (that is, oxygen-carrying proteins contained in cells instead of directly contained in body fluids) appeared , which is an important step in the evolution of vertebrates. It makes the blood still have high oxygen-carrying capacity under low-viscosity conditions, and also enables vertebrates to have a stronger food intake range and ability under the promotion of high oxygen-carrying red blood cells. When vertebrates were evolved to a certain extent, some vertebrates promote their evolution due to predation skills and brain development, so that they can provide a large amount of rich nutrition to maintain a constant body temperature and prolong life. Rich nutrition also promotes the development of mammary glands, making their differentiation from non-mammals to mammals, which has also improved the survival rate of infants and young children, and also made them qualitatively improve the types and scope of food that they could grab and their intelligence. It is precisely because of the rich nutrition that the red blood cells during the period from non-mammals to mammalian undergo apoptosis and enucleation during nuclear replication, evolving from nucleated to enucleated.

Mammals have made great progress in grabbing objects and survival intelligence, etc., and can have sufficient energy security and safety protection. Mammals are the animals with the highest morphological structure and the most complete physiological functions in the animal world. Mammals have relatively developed brains, so they can exhibit more complex behaviors than other animals and can constantly change their behaviors to adapt to the changes in the external environment.

### 4. Platelet Characteristics, Modern Formation Mechanism

Blood platelets are one of the components in mammalian blood. Platelets only exist in mammalian blood, and not in other vertebrates. Platelets are irregular in shape, much smaller than red blood cells and white blood cells, without nuclei. The number of platelets in adult blood is 100−300×10⁹/L. Platelets are disc-shaped, with diameters ranging from 1-4 microns to 7-8 microns , and individual differences are large (5˜12 cubic microns). Because platelets can move and deform, they are polymorphic when observed by general methods.

Modern medicine believes that platelets are part of the cytoplasm, isolated from megakaryocytes in the bone marrow, and then enter the circulatory system. The main functions of platelets are coagulation and hemostasis. When the human body is injured and bleeding, platelets and other coagulation substances in the blood will rush forward in groups in a few seconds, quickly forming clots on the damaged blood vessel wall and agglomerating into groups, forming thrombus, and blocking the damaged wound and blood vessels to stop bleeding and repair broken blood vessels. In some cases, it can also form clots in some atherosclerotic parts of blood vessels, which can easily lead to blood vessel blockage and heart disease. Although similar characteristics of platelets to early mammals once gave mammals an evolutionary advantage, the presence of platelets may have improved the survival rate of early mammals. However, modern medicine believes that the evolution of mammalian platelets can promote the formation of clots in blood vessels and expose humans to the threat of cardiovascular disease. Although birds and other vertebrates do not have platelets, they do have thrombocytes, which are twice the size of platelets and contain a nucleus. Even in mammals, enucleated red blood cells and white blood cells also participate in thrombus formation, but there are more platelets involved in blood coagulation.

After the evolution from non-mammals to mammals, red blood cells were enucleated and platelets were added, so where did the nuclei go? Where do platelets come from? Mammals are susceptible to cardiovascular disease, do birds and other vertebrates get heart disease?

Modern medicine believes that platelets are produced by megakaryocytes. Megakaryocytes are large in size, with huge nuclei, and more and more cytoplasm during growth. When the megakaryocytes gradually mature in the bone marrow and reach a certain degree of maturity, the cytoplasm of the megakaryocytes is lysed and falls off. The fragments of the cytoplasm of these megakaryocytes enter the blood circulation through the blood sinus in the hematopoietic tissue of the bone marrow and become platelets. This is the mechanism by which mammals form platelets in modern medicine.

### 5. New Mechanism of Platelet Formation

Newly formed platelets first pass through the spleen, where about 1/3 are stored. The stored platelets can be freely exchanged with the platelets entering the circulating blood to maintain the normal amount in the blood.

Platelets are small pieces of megakaryotic cytoplasm in the bone marrow that have fallen off. Therefore, they have no nucleus and have a complete cell membrane on the surface. They are in the shape of a biconvex disc, susceptible to mechanical and chemical stimuli, be protrude and appear irregular when being stimulated. Platelets play an important role in the blood clotting process. If the number of platelets in the blood is less than 100,000/μ1 (100×10⁹/L), it is thrombocytopenia, and if it is less than 50,000/μL (50×10⁹/L), there is a risk of bleeding.

The structure of platelets is complex. In short, it has a three-layer structure from outside to inside, that is, the outer layer composed of outer membrane, unit membrane and submembrane microfilament structure is the first layer; the second layer is the gel layer, which is microfilaments and microtubules parallel to the surrounding that can be seen under the electron microscope. the third layer is the micro-organ layer, with structures such as mitochondria, compact bodies, and residual nuclei.

According to this patent, platelets are a type of extremely small cells without nuclei and nucleic acids, or organisms without biologically active nucleic acids. Enucleated red blood cells are cell-like organisms without nuclei and nucleic acids, in principle, it is simpler organisms than prokaryotic cells and viruses. Platelets and enucleated red blood cells should not be called cells because they have no genetic chromosomes, cannot replicate, and cannot synthesize proteins.

This patent believes that in biological evolution, the formation of platelets, enucleation of red blood cells and the differentiation of mammals from non-mammals occur almost simultaneously. It is not that platelets increase the survival rate of mammals, but that these animals have a strong food intake ability, which promotes the evolution of animals into mammals, and provides the survival ability and immunity of such animals, which correspondingly increases the survival rate.

This patent believes that the incidence of cardiovascular diseases in mammals has indeed increased, but the reason for the increase may not be caused by platelets, it may be caused by excessive nutrition that promotes the formation of amyloid from fibrin and glycogen, and may be caused by excessive amyloid promoting platelet formation and frequent occurrence of cardiovascular disease. Because some vertebrates have a stronger food intake ability, they stand out from the vertebrates and evolve into a new species of mammals.

If a platelet is a small piece of cytoplasm that is cleaved from the cytoplasm of a megakaryocyte, it should not have the same composition of cell-like tissue and components, and it should not have a uniform composition of mitochondria, compact bodies, remnant nuclei, and cell-like packets membrane. From the description in this part, platelets are more like a shrunken version of some blood cells, rather than a small piece of cytoplasm that is lysed from the cytoplasm of mature megakaryocytes in the bone marrow. From the perspective of composition, it is most likely the remnant nuclei that fell off when the nucleated red blood cells were lysed and enucleated, and then absorbed and phagocytized by macrophages, transported by macrophages to the blood circulation system, and then shed from macrophages. It looks not cleavage and detachment.

In platelets, there are dense granule contents with extremely high electron density, pyrophosphate, cathepsin, acid hydrolase, fibrinogen, mitochondria, and glycogen granules. Therefore, platelets are not simple cells containing a single substance like red blood cells of merely hemoglobin, but a cell-like organism with complex contents and multiple functions. It can be seen that there is a certain similarity between platelets and cell nuclei.

If platelets are transformed from the nuclei of nucleated red blood cells, where does the DNA, the core component of the nucleus, go?

How long is the lifespan of DNA? According to calculations, the half-life of DNA is about 521 years, that is, the chemical bonds between deoxyribonucleotides will be broken in half every 521 years. Even under the ideal conditions of −5° C., these chemical bonds will be completely decomposed after a maximum of 6.8 million years, so some people say that the lifespan of DNA is 6.8 million years. In reality, when cells die, the remaining enzymes will hydrolyze the chemical bonds among DNA nucleotides, and microorganisms will also accelerate the decomposition process. The most important factor is that DNA can degrade under the affection of water to form phosphate-like organic nitrogen residues.

This patent believes that the dense tubule system (DTS) in platelets, the dense granule content with extremely high electron density and pyrophosphate are likely to be residues formed by the hydrolysis and metabolism of nucleic acids, including DNA and RNA, by residual enzymes after nuclei of nucleated red blood cell are inactivated.

If platelets are the enucleated remnants of nucleated red blood cells, are their numbers relevant?

The average lifespan of red blood cells is 120 days, and that of platelets is 8-9 days (8.5 days on average). Usually, each person has 40 ml of blood cells aging and dying every day. At the same time, there is a corresponding number of new cells. This means that about 1/120 of the total red blood cells are renewed every day, and about 1/8.5 of the total platelets are renewed every day.

Normal range of red blood cell count: adult male 4.0˜5.5×10¹²/L, adult female 3.5˜5.0×10¹²/L, child 4.3˜4.5×10¹²/L, newborn 6.0˜7.0×10¹²/L, the mean of red blood cell concentration: 4×10¹²/L; the daily red blood cell renewal is about 4×10¹²/L/120≈3.33×10¹⁰/L. These ranges may vary from person to person and may also vary from laboratory to laboratory. The platelet count in normal blood is (100˜300)×10⁹/liter (average 200×10⁹/liter), and 1/3 of the platelets are usually stored in the spleen, which means that the platelet concentration is equivalent to 200×(4/3) 10⁹/liter, that is, 266×10⁹/liter. The daily renewal of platelets is about 266×10⁹/L/8.5≈3.13×10¹⁰/L. It can be said that the daily renewal numbers of red blood cells and platelets are basically the same, which further confirms the homology of enucleated red blood cells and platelets.

According to this patent, platelets are the nuclei that are removed from chromosome nuclei during the maturation process of mammalian red blood cells. The removed nuclei are adhered by macrophages and enter the blood system together, or are stored in the spleen for ready use at any time, or are shed from macrophages as platelet monomers, or directly enter the blood system together with enucleated mature red blood cells. Platelets participate in blood cell functions, play the role of blood coagulation. Enucleated red blood cells and platelets are two products derived from nucleated red blood cells during red blood cell formation.

Modern medicine believes that thrombocytopenia may cause prolongation of bleeding time, and bleeding can occur in severe injury or in an excited state. When the platelet count is <50×109/L, mild injury can cause skin and mucous membrane purpura, and bleeding may occur after surgery; when the platelet count is <20×10⁹/L, spontaneous bleeding often occurs. It is generally believed that when the platelet count is <20×10⁹/L, preventive transfusion of platelets is required. If the platelet count is >50×109/L, and the platelet function is normal, there will be no obvious bleeding during the operation.

However, it is increasingly recognized that thrombocytopenia is not as common a cause of clinical bleeding as previously thought. A significant number of patients had bleeding symptoms and their platelet counts were normal.

According to this patent, there are many factors affecting coagulation, including blood cells, platelets, fibrin and blood sugar, and the number of platelets is only one of them. Modern medicine regards platelet concentration as an indicator of blood coagulation ability, but it is only an auxiliary function of blood coagulation, which can be replaced by other blood cells.

The new coagulation mechanism of this patent: when the organism is affected by external damage or toxins, the body's stress response promotes the transformation of fibrinogen into fibrin, and the stress response also promotes the aggregation of glucose, and the fibrin is affected by blood sugar glycogen to form amyloid with low water solubility , amyloid interweaves at the injured site to form a network, when the blood and coagulation factors including blood cells, platelets, proteins, polypeptides and other components flow or permeate through the fibrin network, the coagulation factors and amyloid accumulate on the network to form a coagulation clot to block the damaged part and form a coagulation, and then the coagulation clot is formed induration of hard lumps to seal the wound to achieve hemostasis. If a blood clot occurs in a narrowed place in a blood vessel, it is easy to form a thrombus.

Because red blood cells of inferior animals cannot be enucleated to form platelets and mature red blood cells with special functions, blood coagulation mainly depends on blood cells including nucleated red blood cells to perform coagulation. When mammals, including humans, are injured, both enucleated blood cells and platelets will simultaneously perform coagulation. In the process of blood coagulation, the inferior vertebrates cyclostomum have spindle cells for blood coagulation, and Fishes have specific thrombus cells. There are thrombus cells in the blood of amphibians, reptiles, and birds, which are spindle-shaped or oval cells with nuclei and function similar to platelets. Invertebrates do not have specific thrombus cells, such as amebocytes in molluscs, which have both defense and wound healing functions. Crustaceans have only one type of blood cell, which also has the function of coagulation.

According to this patent, when the concentration of all components or a certain item in blood cells, platelets, fibrin or blood sugar decreases, blood coagulation factors decrease, leading to bleeding symptoms.

Platelet detection can help doctors and patients understand the patient's anemia status. The main function of platelets is blood coagulation, but it is not necessary for blood coagulation. The coagulation function can be replaced by other blood cells.

Finally, this patent believes that during the maturation process of mammalian reticulocytes, enucleated red blood cells are formed, and the red blood cell nuclei are included in the partial cytoplasm. After degeneration, shrinkage and separation, the nuclei are transformed into platelets. Platelets play the role of coagulation and hemostasis, but platelets are not a necessary factor for coagulation and hemostasis. Nucleated red blood cells are not a factor of anemia, but only a manifestation of anemia. Nucleated red blood cells are non-toxic, because of their reproductive ability, they can reduce the hematopoietic load of the organism.

### 6. New Hematopoietic Mechanism

When it comes to artificial blood, blood type must be considered.

The human blood group system includes more than 30 blood group systems such as ABO blood group and Rh blood group, which are mainly based on the antigen types on the surface of blood cells (including red blood cells, white blood cells, and platelets) and antibodies in serum. If the blood antigens and antibodies are inconsistent, hemolysis will occur when the blood is injected into the human body. Therefore, antibody screening and cross-matching are required when transfusing blood products containing red blood cell components. During blood transfusion, the required components of the same blood group antigen or antibody should be infused as much as possible to avoid possible factors of vascular obstruction and massive coagulation and hemolysis in the blood vessel, so as to prevent agglutination reaction and endanger life. Therefore, understanding the different blood group systems is fundamental to clinically safe blood transfusion.

Blood type refers to the type of antigens on the surface of blood components. Modern medicine believes that glycosphingolipids on the plasma membrane of red blood cells are the blood group antigens of the ABO blood group system, and the molecular basis of blood group immune activity specificity is the difference of a single sugar group at the end of the sugar chain.

This patent believes that only one glycosyl difference in red blood cells is not enough to cause huge physiological differences and disasters. Physiologically, polysaccharides have not been found to have antigenic activity, but polysaccharides often have antigenic activity when coupled with proteins. The blood type must be a comprehensive antigen formed mainly by the side chains of red blood cell proteins supplemented by short-chain sugar groups formed due to aging. This patent believes that erythrocytes are crown cells, similar to coronaviruses, and the source of antigens is the protein side chain part on the surface of erythrocytes, especially the protein part that exerts enzyme activity.

Blood type is determined by an enzyme in the body. The gene encoding this enzyme is located on the human chromosome. Since the human chromosome is diploid, one gene comes from the mother and one gene comes from the father. These two genes can be the same or different, so that different protein side chains with enzyme function are derived, and different aged sugar chains are suspended on different protein short chains. These different protein side chains and sugar chains Commonly exhibit different blood types.

Human whole blood is very complex, and artificial whole blood is almost impossible. Artificial blood is not reduced human whole blood, nor does it need to be whole blood, as long as it can contain life-critical components. At present, red blood cells and other blood products mainly come from peripheral blood donations from volunteers, but insufficient donors, infection risks, and lack of rare blood types are still difficult problems in blood transfusion. The artificial blood currently studied is artificially made of some components in blood. There are two ways to realize artificial blood: the first refers to the biosynthesis of red blood cells, white blood cells and platelets with complete biological functions; the other is artificial synthesis of substitute that performs the primary function of red blood cells or platelets.

This patent believes that platelets are an important factor for blood hemostasis and coagulation, but not a necessary factor, and artificial blood does not need to have platelets as a component. Nucleated erythrocytes have a stronger oxygen-carrying function than enucleated erythrocytes, and have the function of replication and heredity, which can increase the self-replication ability and effective number of erythrocytes, improve oxygen supply capacity, reduce the dependence of the bone marrow hematopoietic system, and relieve the impact of bone marrow hematopoiesis in bone marrow diseases. Therefore, there is no need to insist on retaining the procedure of enucleation for artificial blood, which can reduce the production procedures, prepare more powerful nucleated red blood cells, and reduce the time-consuming and cost of artificial blood.

Artificially cultivating blood of different blood types can solve the world-class human problem of difficulty in using blood, especially rare blood types. It can not only eliminate the need for public blood donation, but also avoid the occurrence of infectious diseases. Through in vitro stem cell culture technology, nucleated erythrocytes can be produced in large scale by in vitro blood grouping. Artificial blood can be “genetically modified” to knock out the antigens on the surface of blood cells by gene knockout to make antigen-free universal nucleated red blood cells, so that blood transfusions do not need to be matched again. In this way, the trouble of blood type testing can be avoided, the pressure of blood donation can be relieved, the plight of rare blood type blood supply can be solved, and the life can be saved.

At <−65° C., frozen enucleated red blood cells containing 40% glycerol can be stored for up to 10 years. However, the storage conditions of nucleated red blood cells need further study.

The current bottleneck in the production of artificial blood is the enucleation of nucleated red blood cells to produce so-called mature enucleated red blood cells. Retaining red blood cell nuclei will definitely increase the production speed and scale of red blood cells, greatly reduce costs, and greatly improve the function of red blood cells. It will also solve the centuries-old problem of blood shortage.

What is claimed is:

1. Mitotic Mechanism and Amitotic Mechanism.

2. Mammalian red blood cell enucleation mechanism.

3. Mechanism of cell aging apoptosis.

4. Mechanism of platelet formation. Nucleated erythrocytes are enucleated to form enucleated erythrocytes, and the nuclei removed are transformed into platelets.

5. Coagulation mechanism. Artificial blood does not need to have platelet.

6. Preparation of nucleated erythrocyte artificial blood of different blood types, and genetic engineering universal nucleated erythrocyte artificial blood without antigen.
